(19) United States
(12) Patent Application Publication
(10) Pub. No.: US 2023/0390326 A1
(43) Pub. Date: Dec. 7, 2023

(54) COMPOSITION COMPRISING CATIONIC SUBSTANCE, AND USE FOR SAME

(71) Applicants: SUNGKWANG MEDICAL FOUNDATION, Seoul (KR); CHA UNIVERSITY INDUSTRY-ACADEMIC COOPERATION FOUNDATION, Pocheon-si (KR)

(72) Inventors: Kyung-Soon PARK, Seongnam-si (KR); Keun-Hong PARK, Seoul (KR); Seung Hee CHOI, Namyangju-si (KR); Hye Jin KIM, Suwon-si (KR); Hee Jung AN, Seoul (KR)

(73) Assignees: SUNGKWANG MEDICAL FOUNDATION, Seoul (KR); CHA UNIVERSITY INDUSTRY-ACADEMIC COOPERATION FOUNDATION, Pocheon-si (KR)

(21) Appl. No.: 18/250,876

(22) PCT Filed: Oct. 27, 2021
(86) PCT No.: PCT/KR2021/015147

§ 371 Date: Apr. 27, 2023

(30) Foreign Application Priority Data
Oct. 29, 2020 (KR) 10-2020-0142534

(51) Int. Cl. A61K 31/785; C12N 5/0783; A61P 35/00

(52) U.S. Cl. CPC A61K 31/785; C12N 5/0646; A61P 35/00; C12N 2500/50

(57) ABSTRACT

According to a composition including a cationic substance as an active ingredient according to one aspect, the stability of perforin protein is increased to induce the accumulation of intracellular perforin proteins, thereby increasing the activity of immune cells.

### TECHNICAL FIELD

The present application claims priority to Korean Patent Application No. 10-2020-0142534 filed on Oct. 29, 2020, and the entirety of which is hereby incorporated by reference.

The present disclosure relates to a composition including a cationic substance and use thereof.

### BACKGROUND ART

Natural killer cells (NK cells) are immune cells that perform the primary defense function in the body in the innate immune system. NK cells contain receptors that recognize abnormal cells, and control the immune system by immediately detecting and removing abnormal cells such as cancer cells or viruses without specific antigens, and effectively suppress the proliferation, recurrence, and metastasis of cancer cells.

Using the characteristics of NK cells as described above, research on anti-cancer immunotherapeutic agents using NK cells has recently been actively pursued. Among the methods of immuno-anticancer treatment, the immune cell therapy refers to a therapy in which immune cells in the body are genetically modified using immune cells in the body and then injected back into the body. Immunotherapy aims to enhance cellular immunity, but chimeric antigen receptor T-cells (CAR-T cells) have the disadvantages of complex genetic manipulation and high treatment costs, and the characteristics of T cells led to cytokine release syndrome and other side effects. In order to overcome these issues, NK cells are newly attracting attention in the field of immune-anticancer treatment.

The present inventors solved these issues by confirming that stabilization of perforin protein was increased when immune cells were treated with a cationic substance.

### DETAILED DESCRIPTION OF THE DISCLOSURE

### Technical Problem

One aspect provides a composition for increasing intracellular perforin protein, the composition including a cationic substance as an active ingredient.

Another aspect provides a composition for enhancing an activity of immune cells, the composition including a cationic substance as an active ingredient.

Another aspect provides immune cells of which the perforin expression or amount is increased relative to normal cells due to the pretreatment with a cationic substance.

Another aspect provides a composition for preventing or treating cancer, the composition including immune cells of which the perforin expression or amount is increased relative to normal cells due to the pretreatment with a cationic substance.

Another aspect provides a pharmaceutical composition for preventing or treating cancer, the pharmaceutical composition including a cationic substance and immune cells as active ingredients.

Another aspect provides a culture method of promoting an activity of immune cells, the culture method including culturing the immune cells with a cationic substance.

Another aspect provides a method of increasing an amount or expression level of perforin in immune cells, the method including culturing immune cells with a cationic substance.

Another aspect provides a method of preventing or treating cancer, the method including administering to a subject a composition including a cationic substance and immune cells as active ingredients.

Another aspect provides use of a composition including a cationic substance as an active ingredient for use in increasing intracellular perforin protein.

Another aspect provides use of a composition including a cationic substance as an active ingredient for use in increasing an activity of immune cells.

Another aspect provides use of a composition including a cationic substance as an active ingredient for use in the manufacture of a medicament for preventing or treating cancer.

Another aspect provides use of a cationic substance for use in increasing intracellular perforin protein.

### Technical Solution to Problem

One aspect provides a composition for increasing intracellular perforin protein, the composition including a cationic substance as an active ingredient.

Another aspect provides a composition for enhancing an activity of immune cells, the composition including a cationic substance as an active ingredient.

The term “cationic substance” used herein may be a substance having a positive ion on the surface thereof, or a substance having a positive charge on the surface thereof.

In an embodiment, the cationic substance may be polyethylenimine (PEI) or chitosan. In an embodiment, the cationic substance may be polyethylenimine.

The term “polyethylenimine (PEI)” used herein is a polymer having the formula of (C₃₇H₂₄O₆N₂), and has a density of 1.27 g/cm³. The polyethylenimine may be a cationic substance. The cationic property of the polyethylenimine may enable the transfer of genes into cells.

The cells may be immune cells, and may be T cells, B cells, dendritic cells, or natural killer cells.

The enhancement of the activity of immune cells indicates increased immunomodulatory, cytotoxic, or apoptotic capacity of a cell relative to a parent cell, e.g., a hematopoietic cell, or a progenitor cell. The immune cells may be CAR-immune cells.

The term “natural killer cell (NK cell)” is a type of white blood cell in the blood responsible for immunity, and refers to a cell that matures in the liver and bone marrow. The NK cells are responsible for non-specific immunity and may remove viruses, cancer cells, and the like.

In an embodiment, treatment of NK cells with the composition results in increased immunomodulatory, cytotoxic, or apoptotic capacity of the natural killer cells. Therefore, it was confirmed that due to the inclusion of the composition including natural killer cells with increased immunomodulatory, cytotoxic, or apoptotic capacity, the ability of NK cells to kill viruses or cancer cells is increased.

The term “perforin protein” refers to a glycoprotein that destroys cells by creating pores in the plasma membrane of cells. The cellular perforin protein may be present in immune cells.

The polyethylenimine may have a branched structure. The branching refers to a chemical structure that is not linear and includes branches. In addition, the polyetheramine may be a primary, secondary or tertiary amine.

The molecular weight of the polyethylenimine may be 10,000 mM to 30,000 mM. For example, the molecular weight of the polyethylenimine may be 10,000 mM to 28,000 mM, 10,000 mM to 27,000 mM, 12,000 mM to 30,000 mM, 12,000 mM to 28,000 mM, 12,000 mM to 27,000 mM, 15,000 mM to 30,000 mM, 15,000 mM to 28,000 mM, or 15,000 mM to 27,000 mM. In an embodiment, the molecular weight of the polyethylenimine may be about 25,000 mM. Since gene transfer efficiency is increased in proportion to the density of cations, when the range of the molecular weight is greater than or less than this value, gene transfer efficiency may be reduced.

The polyethylenimine may be branched or linear. In addition, the branched type may have at least one selected from the group consisting of primary, secondary and tertiary amines in one molecule. The branched type has one or more amine structures in one molecule and within a wide pH range, may have a proton sponge effect capable of changing into cations.

In an embodiment, the amount of the cationic substance may be 0.1 μg/ml to 10 μg/ml. For example, the amount of the cationic substance may be 0.1 μg/ml to 9 μg/ml, 0.1 μg/ml μg/ml to 8 μg/ml, 0.5 μg/ml to 10 μg/ml, 0.5 μg/ml to 9 μg/ml, 0.5 μg/ml to 8 μg/ml, 1 μg/ml to 10 μg/ml, 1 μg/ml to 9 μg/ml, 1 μg/ml to 8 μg/ml, 2 μg/ml to 10 μg/ml, 2 μg/ml to 9 μg/ml, 2 μg/ml to 8 μg/ml, 3 μg/ml to 10 μg/ml, 3 μg/ml to 9 μg/ml, or 3 μg/ml to 8 μg/ml. At this time, when the amount of the cationic substance is less than or greater than this range, the immune cells may not be sufficiently activated or the accumulation of perforin protein may be reduced.

In an embodiment, the composition may be a medium composition. The composition for increasing perforin protein or the composition for promoting immune cell activity may, when incubated with immune cells in a medium composition, induce stabilization or accumulation of perforin protein in the immune cells and, consequently, activation of the immune cells.

The composition may further include nanoparticles.

The term “nanoparticles” used herein may refer to particles having a surface and having a size of 1 nm to 100 nm. The nanoparticles may be coated.

In an embodiment, the nanoparticles may be magnetic nanoparticles. In an embodiment, the core of the nanoparticle may contain Zn or Fe. The nanoparticles may have magnetism due to the core layer.

The cationic substance may be bound to nanoparticles. Specifically, the cationic substance may be present on the surface of the nanoparticle or may be chemically bonded to the surface of the nanoparticles. Regarding the composition according to an embodiment, by culturing the nanoparticles and the cationic substance for a certain period of time, polyetheramine is bound to the surface of the nanoparticles, and a composition including the nanoparticles and the cationic substance may be obtained.

In an embodiment, the composition may induce accumulation of intracellular perforin protein by stabilizing perforin protein. The stabilization may indicate resistance to proteolytic degradation leading to an increase in the amount of perforin protein and improvement in translation efficacy from perforin mRNA. Accordingly, the composition may increase the number or amount of perforin proteins in cells.

The composition may be administered simultaneously with an immuno-oncology agent. When co-administered with an immuno-oncology agent, the composition may increase the activity of the immuno-oncology agent.

The composition may further include at least one selected from the group consisting of gamma-PGA, glycol chitosan, and protamine.

Another aspect provides immune cells of which the perforin expression or amount is increased relative to normal cells due to the pretreatment with a cationic substance.

Another aspect provides a composition for preventing or treating cancer, the composition including the immune cells.

The cationic substance, cells, perforin, and immune cells are the same as described above.

The composition for preventing or treating cancer may be an immuno-cancer agent.

The term “immuno-oncology agent” may refer to an anti-cancer agent that kills cancer cells by activating the body's immune cells, or an agent that has a therapeutic effect on cancer by enhancing the patient's own immunity. In an embodiment, the immuno-oncology agent may be a therapeutic agent for immune cells.

The term “immune checkpoint inhibitor” refers to an immuno-oncology agent that activates T cells to attack cancer cells by blocking the activation of an immune checkpoint protein involved in the suppression of T cells, such as a protein such as PD-L1 expressed on tumor cells.

In an embodiment, the immune checkpoint inhibitor may be at least one selected from the group consisting of NK checkpoint inhibitors, T checkpoint inhibitors, CAR-immune checkpoint inhibitors, DC vaccines, CTL therapeutics, anti-PD-L1, anti-PD-1, and anti-CTLA-4. In an embodiment, the immune checkpoint inhibitor may be an NK checkpoint inhibitor.

In an embodiment, the CAR-immune checkpoint inhibitor may indicate an immune checkpoint inhibitor including chimeric antigen receptor-T (CAR T) or chimeric antigen receptor-NK (CAR-NK) cells.

Another aspect provides a method of treating cancer in a subject including administering the pharmaceutical composition to the subject.

Another aspect provides a method of preventing or treating cancer including administering to a subject a composition including a cationic substance as active ingredients.

Details of the cationic substance, polyetheramine or immune cells are the same as described above.

The term “subject” used herein refers to a subject in need of cancer treatment, and more specifically, a human or non-human primate, or mammals, such as mouse, rat, dog, cat, horse, or cow. The cancers may include at least one selected from the group consisting of breast cancer, thyroid cancer, stomach cancer, colon cancer, lung cancer, liver cancer, prostate cancer, pancreatic cancer, gallbladder cancer, biliary tract cancer, non-Hodgkin's lymphoma, oral cancer, oral cancer, testicular cancer, acute myelogenous leukemia, basal cell cancer, ovarian epithelial cancer, brain tumor, multiple myeloma, hematological cancer, chronic myelogenous leukemia, chronic lymphocytic leukemia, bladder cancer, peritoneal cancer, tongue cancer, non-small cell lung cancer, small cell lung cancer, small bowel cancer, esophageal cancer, kidney cancer, heart cancer, malignant lymphoma, urethral cancer, cervical cancer, rectal cancer, tonsillar cancer, and laryngeal cancer.

The term “prevention” refers to any act of inhibiting or delaying the development of cancer by administration of a composition according to the present disclosure.

The term “treatment” refers to, or includes, the alleviation, arrest of progression, or prevention of a disease, disorder, or pathology, or one or more symptoms thereof, and the term “active ingredient” or the term “pharmaceutically effective amount” may refer to an amount of a composition used in the process of practicing the present disclosure provided herein that is sufficient for the alleviation, arrest of progression, or prevention of a disease, disorder, or pathology, or one or more symptoms thereof.

Since the pharmaceutical composition includes, as an active ingredient, an immune cell in which an amount of perforin is increased due to a cationic substance so that immune responses thereof are activated, the pharmaceutical composition may be effectively used for the treatment of cancer.

The composition may further include other known immune adjuvants, and other immune adjuvants may include one of monophosphoryl lipid A (MPL) and GLA-SE (Glucopyranosyl Lipid Adjuvant, formulated in a stable nano-emulsion of squalene oilin-water).

The administration method of the pharmaceutical composition is not particularly limited, and may be administered orally or parenterally, such as intravenous, subcutaneous, intraperitoneal, inhalation or topical application, depending on the desired method. The dosage varies depending on the weight, age, sex, health condition, diet, administration time, administration method, excretion rate, and severity of the disease, of the patient. A daily dose refers to an amount of a therapeutic substance according to one aspect sufficient to treat a disease state alleviated by being administered to a subject in need thereof. An effective amount of a therapeutic agent may vary depending on the particular compound, the disease state and severity thereof, and the subject in need of treatment, and may be routinely determined by a person skilled in the art. As a non-limiting example, the dosage of the composition according to one aspect to the human body may vary depending on the age, weight, and sex of the patient, dosage form, state of health, and degree of disease. Regarding an adult patient weighing 70 kg, for example, about 1,000 cells/dose to about 10,000 cells/dose, about 1,000 cells/dose to about 100,000 cells/dose, about 1,000 cells/dose to about 1000,000 cells/dose, about 1,000 cells/dose to about 10,000,000, about 1,000 cells/dose to about 100,000,000 cells/dose, about 1,000 cells/dose to about 1,000,000,000 cells/dose, or about 1,000 cells/dose to about 10,000,000,000 cells/dose may be administered once or several times daily in divided doses at regular time intervals, or multiple times at regular time intervals.

The pharmaceutical composition may include pharmaceutically acceptable carriers and/or additives. For example, sterile water, physiological saline, common buffers (phosphoric acid, citric acid, other organic acids, etc.), stabilizers, salts, antioxidants (ascorbic acid, etc.), surfactants, suspending agents, tonicity agents, or preservatives, etc., may be included. For topical administration, a combination thereof with organic substances such as biopolymers, inorganic substances such as hydroxyapatite, for example, collagen matrices, polylactic acid polymers or copolymers, polyethylene glycol polymers or copolymers, and chemical derivatives thereof, may be included. When the pharmaceutical composition according to an embodiment is prepared in a formulation suitable for injection, the immune cells or substances that increase the activity thereof may be dissolved in a pharmaceutically acceptable carrier or frozen in a dissolved solution state.

The pharmaceutical composition, when necessary according to the administration method or dosage form, may appropriately include a suspending agent, a solubilizing agent, a stabilizing agent, an isotonic agent, a preservative, an adsorption preventing agent, a surfactant, a diluent, an excipient, a pH adjusting agent, a pain reliever, a buffer, a reducing agent, an antioxidant, and the like. Pharmaceutically acceptable carriers and agents suitable for the present disclosure, including those exemplified above, are described in detail in Remington's Pharmaceutical Sciences, 19th ed., 1995. The pharmaceutical composition may be prepared in unit dosage form, or prepared by placing the same in a multi-dose container, by formulation using a pharmaceutically acceptable carrier and/or excipient according to a method that may be easily performed by a person skilled in the art. The dosage form may be in the form of a solution, suspension or emulsion in an oil or aqueous medium, or in the form of a powder, granule, tablet or capsule.

Another aspect provides a culture method of promoting the activity of immune cells, the method including culturing the immune cells with a cationic substance.

Another aspect provides a method of increasing the amount or expression level of perforin in immune cells, the method including culturing the immune cells with a cationic substance.

Details of the cationic substance, polyethylenimine, immune cells, and promotion of activity are the same as described above.

The culturing process may be culturing a cationic substance and immune cells for 5 hours to 60 hours. For example, the culturing may be performed for 5 hours to 60 hours, 12 hours to 60 hours, 6 hours to 58 hours, 6 hours to 55 hours, 6 hours to 53 hours, 6 hours to 50 hours, 8 hours to 60 hours, 8 hours to 58 hours, 8 hours to 55 hours, 8 hours to 53 hours, 8 hours to 50 hours, 10 hours to 60 hours, 10 hours to 58 hours, 10 hours to 55 hours, 10 hours to 53 hours, or 10 hours to 50 hours. When the culturing time is greater than or less than these ranges, stabilization of perforin may not be sufficiently achieved.

According to the method, by culturing the immune cells with a cationic substance, the stabilization of perforin protein is increased and intracellular accumulation is induced, thereby enhancing apoptosis.

Another aspect provides use of a composition including a cationic substance as an active ingredient for use in increasing intracellular perforin protein.

Another aspect provides use of a composition including a cationic substance as an active ingredient for use in increasing the activity of immune cells.

Another aspect provides use of a composition including a cationic substance as an active ingredient for use in the manufacture of a medicament for preventing or treating cancer.

Another aspect provides use of the cationic substance for use in increasing intracellular perforin protein.

The meaning of terms such as the cationic substance, polyethylenimine, immune cell, activation promotion, subject, administration, prevention or treatment may be the same as described above.

Advantageous Effects of Disclosure According to the composition including a cationic substance as an active ingredient according to one aspect, the stability of perforin protein is increased to induce the accumulation of intracellular perforin protein, thereby increasing the activity of immune cells.

### BRIEF DESCRIPTION OF DRAWINGS

FIG. 1 is a schematic diagram showing the results of treating natural killer cells with polyethylenimine.

FIG. 2A to FIG. 21 are a diagram showing the activity of natural killer cells as a result of treating natural killer cells with branched or linear polyethylenimine.

FIG. 3 is a diagram showing the activity of natural killer cells as a result of treating breast cancer cells with different concentrations of 25K molecular weight polyethylenimine.

FIG. 4 is a graph showing the quantification of the activity of natural killer cells as a result of treating breast cancer cells with different concentrations of 25K molecular weight polyethylenimine.

FIG. 5A to FIG. 5C are a diagram showing the degree of apoptosis of breast cancer cells as a result of culturing at various effector (E):target (T) ratios of breast cancer cells with 5 μg/ml of 25K molecular weight polyethylenimine.

FIG. 6 is a diagram showing the quantification of the degree of apoptosis of breast cancer cells as a result of culturing at various E:T ratios of breast cancer cells with 5 μg/ml of 25K molecular weight polyethylenimine.

FIG. 7A to 7D are a diagram showing the activity of natural killer cells as a result of treating natural killer cells with polyethylenimine of which the cationic property had been inhibited.

FIG. 8 is a diagram showing the quantification of the activity of natural killer cells as a result of treating natural killer cells with polyethylenimine of which the cationic property had been inhibited.

FIG. 9 is a graph showing changes in the expression of granzyme protein as a result of culturing natural killer cells with 5 μg/ml of polyethylenimine over time.

FIG. 10 is a graph showing changes in the expression of perforin protein as a result of culturing natural killer cells with 5 μg/ml of polyethylenimine over time.

FIG. 11 is an image showing changes in expressions of granzyme protein and perforin protein as a result of culturing natural killer cells with 5 μg/ml of polyethylenimine over time.

FIG. 12 is an image showing changes in the expression of granzyme protein and perforin protein as a result of culturing natural killer cells with 5 μg/ml of polyethylenimine for 48 hours.

FIG. 13 is an image showing changes in expression of perforin protein as a result of culturing natural killer cells with polyethylenimine and MG132.

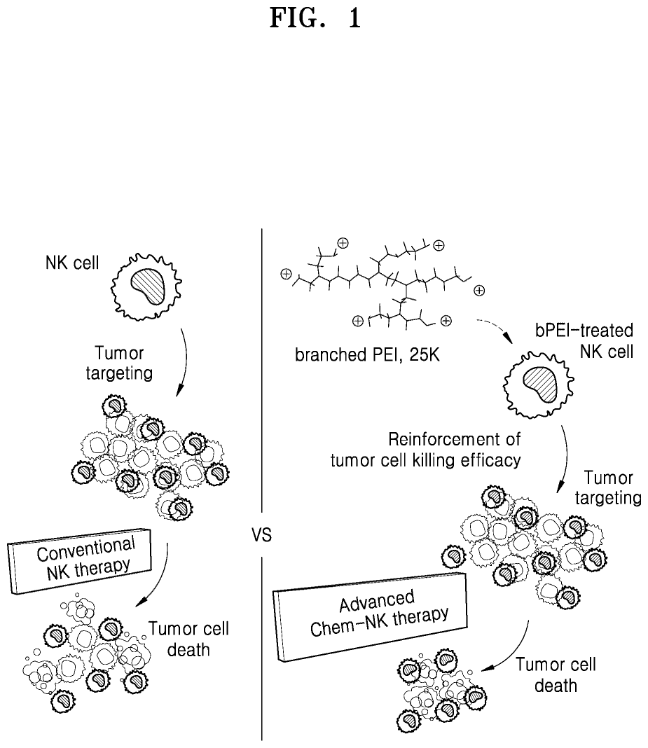

### MODE OF DISCLOSURE

Hereinafter, the present disclosure will be described in more detail through examples. However, these examples are intended to illustrate the present disclosure, and the scope of the present disclosure is not limited to these examples.

### Example 1. Preparation of Materials and Animal Models

### 1.1 Process of Preparing Compound or Manufacturer Therefor

Polyethylenimine in branched form (Sigma-Aldrich, Sigma 408727) was diluted to 5 mg/ml in distilled water and used according to the capacity.

### 1.2 Preparation of Natural Killer (NK) Cells

12.5% FBS (gibco 16000-044), 1% P/S (gibco15140-122), 2 mM L-glutamine (gibco25030-081), 0.2 mM inositol (Sigma 17508), 0.1 mM 2-mercaptoethanol, 0.02 mM folic acid (Sigma F8785) were uniformly diluted in Alpha-MEM media (gibco12561-056) media base, and then sterilized using a filter (corning 430758) before use. NK cells were cultured in a T75 flask at 3×10⁵/ml in a 37° C. CO₂ 5% incubator.

### 1.3 In Vitro Assay Method of Inducing Activity of Natural Killer Cells

Green fluorescence-labeled MDA MB 231 cells and NK cells were put into a 1.5 ml eppendorf tube in respective ratios, mixed, and reacted in a 37° C., CO₂ 5% incubator for 4 hours. After the reaction was over, the cells were stained with 7AAD (Invitrogen A1310) for 20 minutes and fixed. Through flow cytometry, the mixed cells were divided into two cell populations according to the presence or absence of green fluorescence, and the percentage of dead cells was measured and compared by group to analyze the activity.

### 1.4 Western Blot Analysis Method

Protein extracts from NK cells were separated using 10% SDS-PAGE, and transferred to a polyvinylidene difluoride amersham Biosciences membrane for 90 minutes at the voltage of 120 V. The membrane was blocked in 3% bovine serum albumin (BSA)-containing tris-buffered saline-Tween [TBST; 0.2 M NaCl, 0.1% Tween-20, and 10 mM Tris (pH 7.4)] for 1 hour. The blocked membrane was incubated with a rabbit polyclonal anti-Perforin antibody (1:1000; ab180773, abcam) or a rabbit monoclonal anti-GAPDH antibody (1:1000; 3683S, Cell signaling). After incubation, the membrane was incubated with anti-rabbit polyclonal IgG (1:5000; #7074, Cell Signaling Technology) for 1 hour at room temperature. After each step, the membrane was washed several times using TBST and bound antibodies were detected using an enhanced chemiluminescence detection system (Thermo Fisher Scientific Biosciences) according to manufacturer instructions

### Example 2. Confirmation of NK Cell Activity Inducing Ability by Structure of Polyetheramide

In order to confirm the ability of polyetheramine to induce the activity of NK cells according to molecular weight and type, the NK cells of Example 1 were cultured for 48 hours after mixing with various molecular weights and types of polyethylenimine or with a control group.

The molecular weight, charge, and structure of various molecular weights and types of polyethylenimine or a comparative compound, which were used in the present experiment, are shown in Table 1.

Specifically, as shown in Table 1 below, branched polyethylenimine with a molecular weight of 1.8 K, branched polyethylenimine with a molecular weight of 10 K, linear polyethylenimine with a molecular weight of 25 K, branched polyethylenimine with a molecular weight of 25 K, gamma-PGA with a molecular weight of 750 K, glycol chitosan with a molecular weight of 5 K, and protamine with a molecular weight of 4.5K were mixed with NK cells and cultured for 48 hours. As the branched polyetheramines, primary, secondary and tertiary amines were used, and the preceding polyetheramines used were secondary amines.

TABLE 1

| | Compound | Molecular | | Structure of |
|---|---|---|---|---|
| No. | name (Mw) | Weight | Charge | amine |
| 1 | Branched PET (25K) | 25,000 | (+ + +) | Primary, secondary |
| | | | | and tertiary |
| 2 | Linear PEL (25K) | 25,000 | (+) | Secondary |
| 3 | Branched PET (10K) | 10,000 | (+) | Primary, secondary |
| | | | | and tertiary |
| 4 | Branched PET (1.8K) | 1,800 | (+) | Primary, secondary |
| | | | | and tertiary |
| 5 | Gamma-PGA | 750,000 | (−) | — |
| 6 | Glycol Chitosan | 5,000 | (+) | Primary and |
| | | | | secondary |
| 7 | Protamine | 4,500 | (+) | Primary and |
| | | | | secondary |

Next, the cultured NK cells were washed, and re-suspended in a fresh culture medium. Thereafter, the NK cells and triple negative breast cancer cell line MDA_MB231 were mixed in an effector (E):target (T) ratio of 10:1 and cultured for 4 hours. Finally, the degree of apoptosis of the target cells in the cultured cells was quantitatively analyzed using the CFSE-7AAD assay.

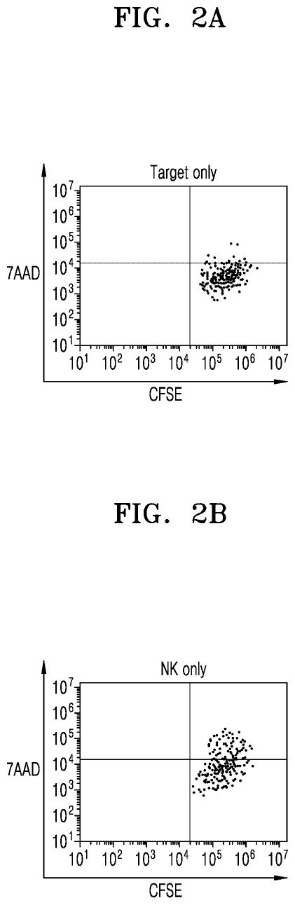

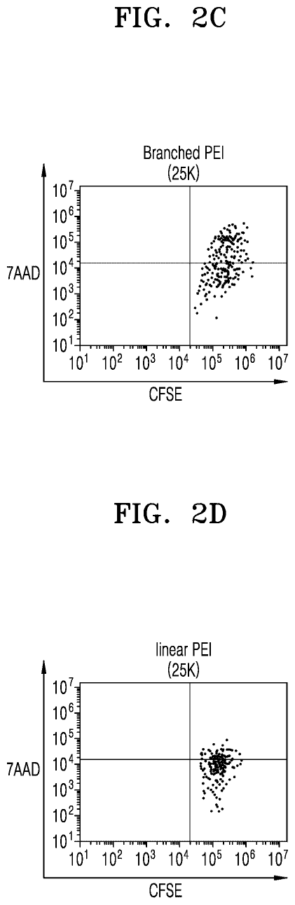

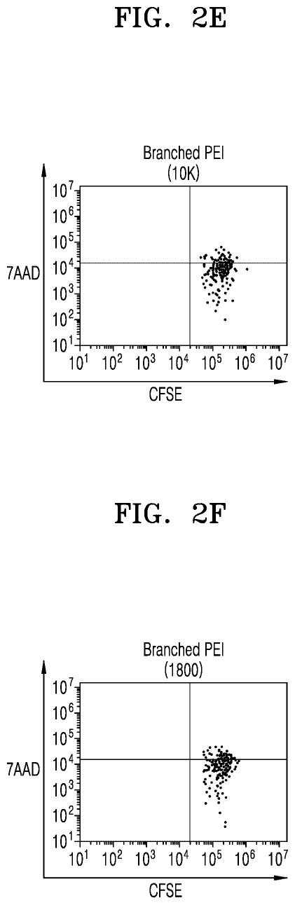

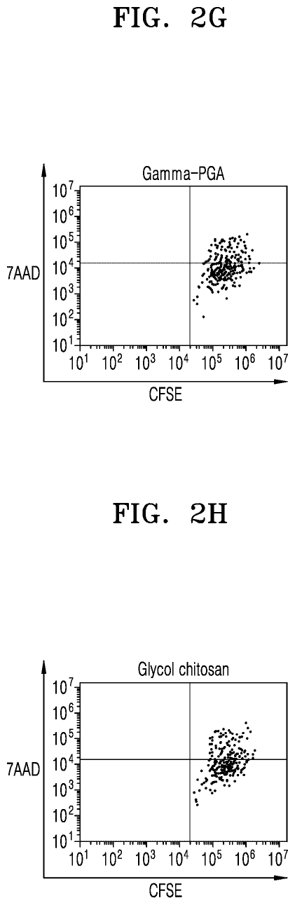

FIG. 2 is a diagram showing the activity of natural killer cells as a result of treating natural killer cells with branched or linear polyethylenimine.

As a result, as shown in FIG. 2, the degrees of apoptosis were low as follows: (1.32) in the case where there was only a target to be compared; (21.36) in the case where only NK cells were present, (22.99) in the case where a branched polyethylenimine with a molecular weight of 1.8 K was mixed, (24.07) in the case where a branched polyethylenimine with a molecular weight of 10 K was mixed, (26.12) in the case where linear polyethylenimine with a molecular weight of 25 K was mixed, (23.08) in the case where gamma-PGA with a molecular weight of 750K was mixed, (32.14) in the case where glycol chitosan with a molecular weight of 5 K was mixed, and (25.31) in the case where protamine with a molecular weight of 4.5 K was mixed. On the other hand, when NK cells were cultured with branched polyethylenimine with a molecular weight of 25K (Branched PET (25K), (59.28)), the degree of apoptosis of triple negative cancer cells was higher than that of the control group. These results indicate that the use of branched polyethylenimine of 10K or more enhances the activity of NK cells compared to other compounds.

### Example 3. Confirmation of Cancer Cell Killing Ability According to Concentration of Polyethylenimine

In order to confirm the ability of polyetheramine to induce the activity of NK cells according to concentration, the NK cells of Example 1 were treated with polyethylenimine with a molecular weight of 25K having different concentrations (0 μg/ml, 0.63 μg/ml, 1.25 μg/ml, 2.5 μg/ml, or 5 μg/ml) and then cultured for 48 hours.

Next, the cultured NK cells were washed, and re-suspended in a fresh culture medium. Thereafter, the NK cells and triple negative breast cancer cell line MDA_MB231 were mixed in an effector (E):target (T) ratio of 10:1 and cultured for 4 hours. Finally, the degree of apoptosis of the target cells in the cultured cells was quantitatively analyzed using the CFSE-7AAD assay.

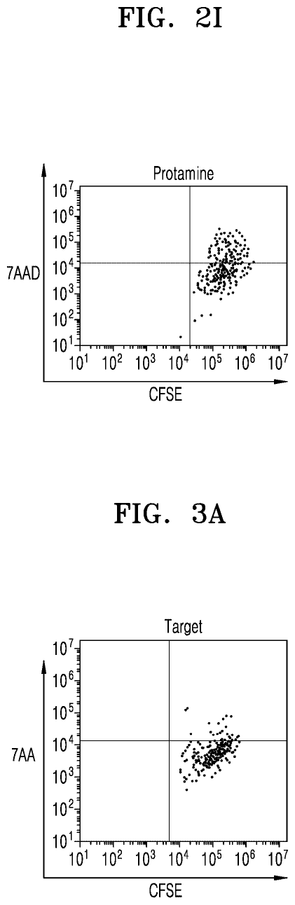

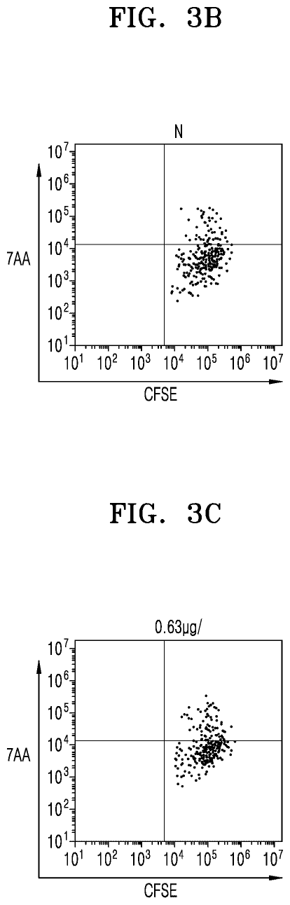

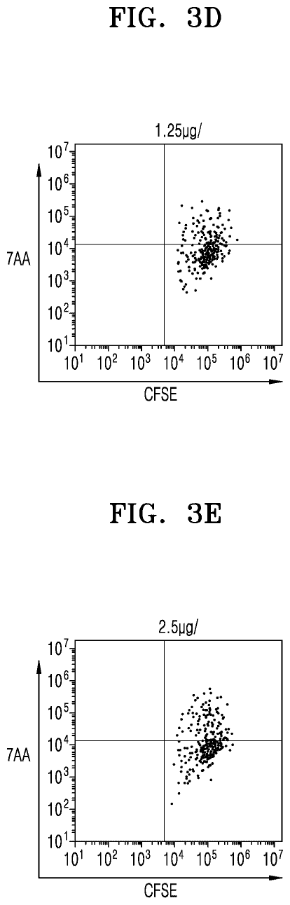

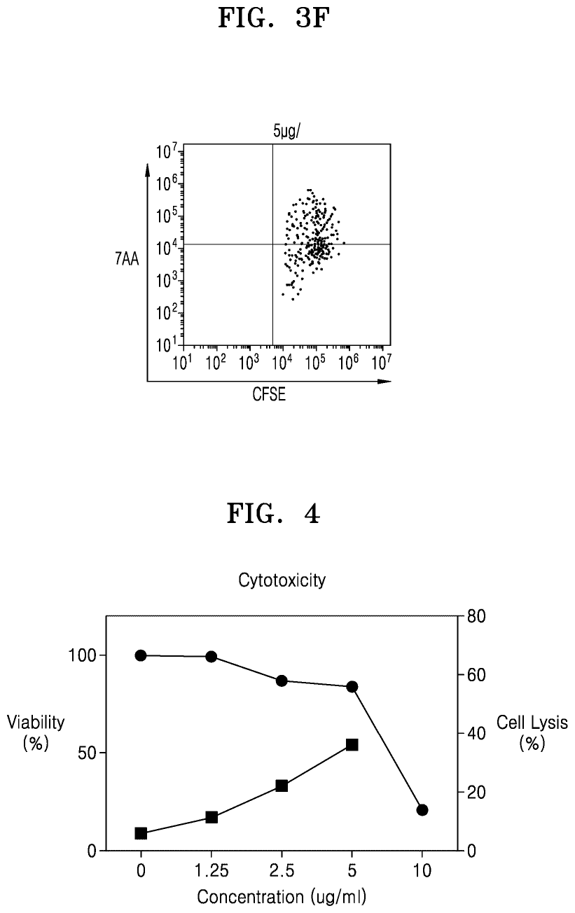

FIG. 3 is a diagram showing the activity of natural killer cells as a result of treating breast cancer cells with different concentrations of 25K molecular weight polyethylenimine. The results show the degrees of activities in various cases including a case where only the target to be compared was present (5.0), a case where there was no target (N, 9.1), a case where the cells were treated at a concentration of 0.63 μg/ml (14.6), a case where the cells were treated at a concentration of 1.25 μg/ml (24.2), a case where the cells were treated at a concentration of 2.5 μg/ml (32.8), and a case where the cells were treated at a concentration of 5 μg/ml (55.1).

FIG. 4 is a graph showing the quantification of the activity of natural killer cells as a result of treating breast cancer cells with different concentrations of 25K molecular weight polyethylenimine.

As a result, as shown in FIGS. 3 and 4, it was confirmed that as the concentration of polyethylenimine increases, the apoptosis increases, and at a concentration of 5 μg/ml, while maintaining 90% or more of the NK cell activity, the immune activity is the highest, and at a concentration of 10 μg/ml or more, the apoptosis is significantly reduced.

### Example 4. Confirmation of Apoptosis Depending on Ratios of Polyethylenimine and Breast Cancer Cells

In order to confirm the cancer cell killing ability according to a mixing ratio of polyetheramine and cancer cells, the NK cells of Example 1 were treated with polyethylenimine having a molecular weight of 25K and 5 μg/ml at each concentration, and the treated NK cells ad triple negative breast cancer cells MDA-MB231 were cultured at E:T ratios (1.25:1, 2.5:1, 5:1, or 10:1) for 48 hours.

Next, the cultured NK cells were washed, and re-suspended in a fresh culture medium. Thereafter, the NK cells and triple negative breast cancer cell line MDA_MB231 were mixed in an effector (E):target (T) ratio of 10:1 and cultured for 4 hours. Finally, the degree of apoptosis of the target cells in the cultured cells was quantitatively analyzed using the CFSE-7AAD assay.

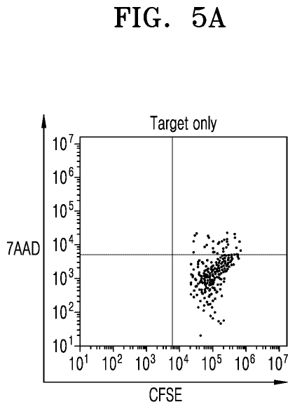

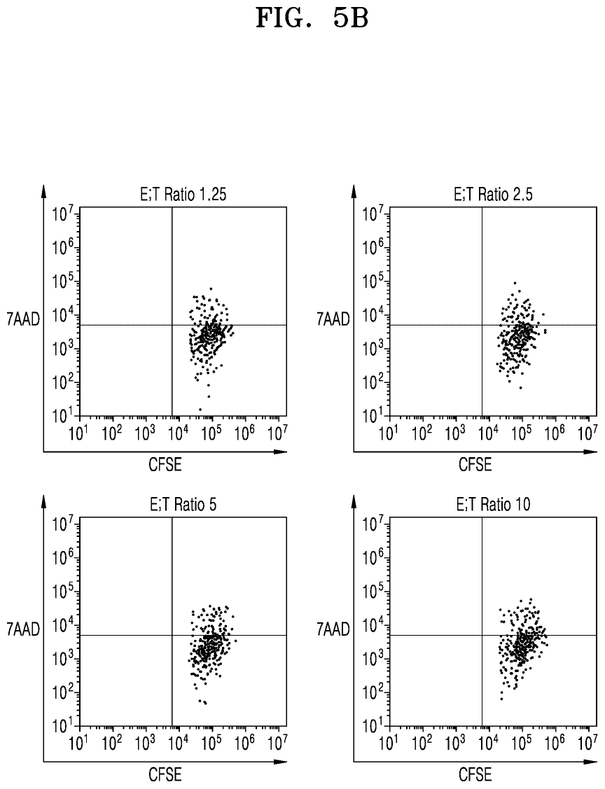

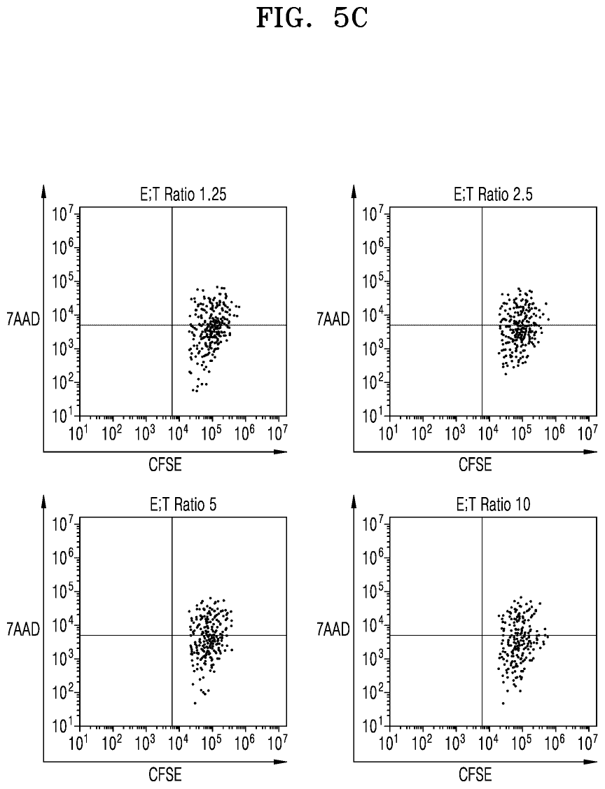

FIG. 5 is a diagram showing the degree of apoptosis of breast cancer cells as a result of culturing at various E:T ratios of breast cancer cells with 5 μg/ml of 25K molecular weight polyethylenimine. When there was only a target to be compared, the value was 2.58. The non-treatment group showed 9.80 for the E:T ratio of 1.25, 9.53 for the E:T ratio of 2.5, 11.92 for the E:T ratio of 5, and 17.56 for the E:T ratio of 10. The group treated with PEI at a concentration of 5 μg/ml showed 36.55 for the E:T ratio of 1.25, 39.90 for the E:T ratio of 2.5, 43.82 for the E:T ratio of 5, and 49.55 for the E:T ratio of 10.

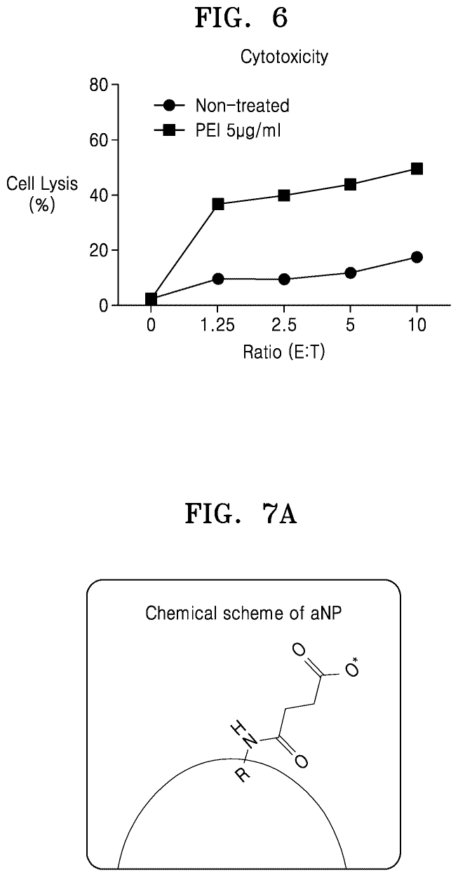

FIG. 6 is a diagram showing the quantification of the degree of apoptosis of breast cancer cells as a result of culturing at various E:T ratios of breast cancer cells with 5 μg/ml of 25K molecular weight polyethylenimine.

As a result, as shown in FIGS. 5 and 6, the activity of natural killer cells at all E:T ratios was higher than that of the untreated group, and in particular, the case where the E:T ratio was 10:1 was the best.

### Example 5. Cation-Dependent Analysis of Polyethylenimines

In order to confirm the ability of polyetheramine to induce the activity of NK cell against electric charges, the nanoparticles of Example 1 were coated with hyalunic acid, which has an anionic property.

Specifically, cationic polyethylenimine was first bound to Zn/Fe nanoparticles by electric interaction, and then anionic hyalunic acid was bound to cationic polyethylenimine thereon, and the zeta potential of the nanoparticles was measured to confirm the anionic property.

Next, the cultured NK cells were washed, and re-suspended in a fresh culture medium. Thereafter, the NK cells and triple negative breast cancer cell line MDA_MB231 were mixed in an effector (E):target (T) ratio of 10:1 and cultured for 4 hours. Finally, the degree of apoptosis of the target cells in the cultured cells was quantitatively analyzed using the CFSE-7AAD assay.

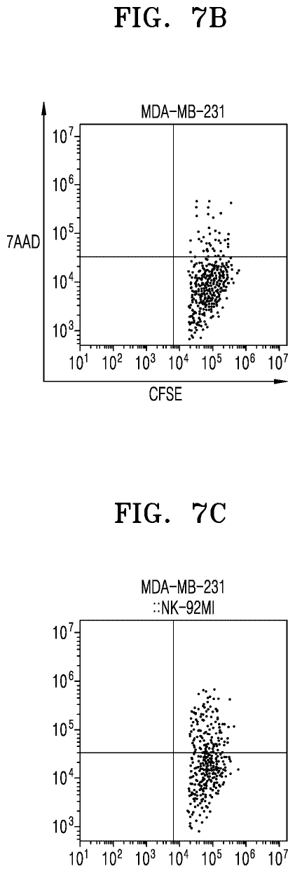

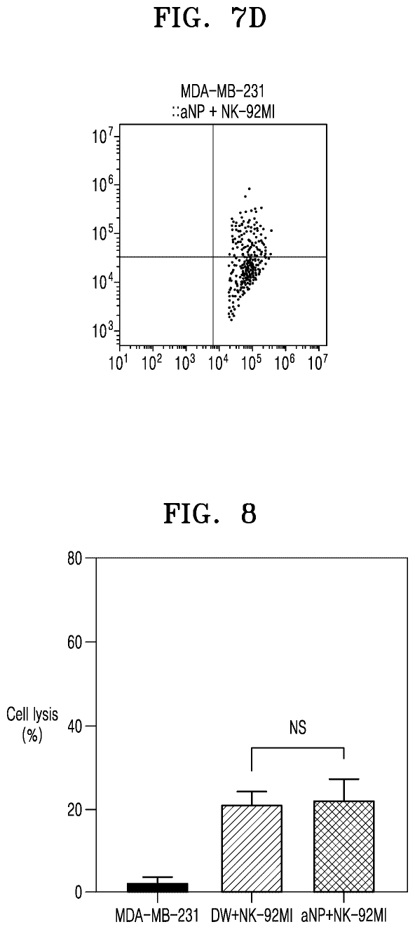

FIG. 7 is a diagram showing the activity of NK cells as a result of treating natural killer cells with polyethylenimine of which the cationic property had been inhibited. The values were 1.02 for MDA-MB-231, 18.47 for MDA-MB-231 and NK-92M1, and 18.21 for MDA-MB-231 and NK-92MI and aNP.

FIG. 8 is a diagram showing the quantification of the activity of natural killer cells as a result of treating NK cells with polyethylenimine of which the cationic property had been inhibited.

As a result, as shown in FIGS. 7 and 8, when the cationic property was reduced by the anionic coating, it was found that the activation ability of NK cells was inhibited. These results indicate that the NK cell activating ability of polyetheramine is dependent on the cationic property.

### Example 6. Analysis of Perforin Protein Stabilization Ability of Polyethylenimine

### 6.1 Analysis of Stabilization Ability by Culturing Time

In order to determine whether polyetheramine increases the amount of intracellular perforin, the NK cells of Example 1 were treated with 5 μg/ml of polyethylenimine, and cultured over time (0 hour, 3 hour, 6 hour, 12 hours, 24 hours, or 48 hours), and then Western blotting was performed on the cultured NK cells to identify the amount of granzyme B and perforin protein.

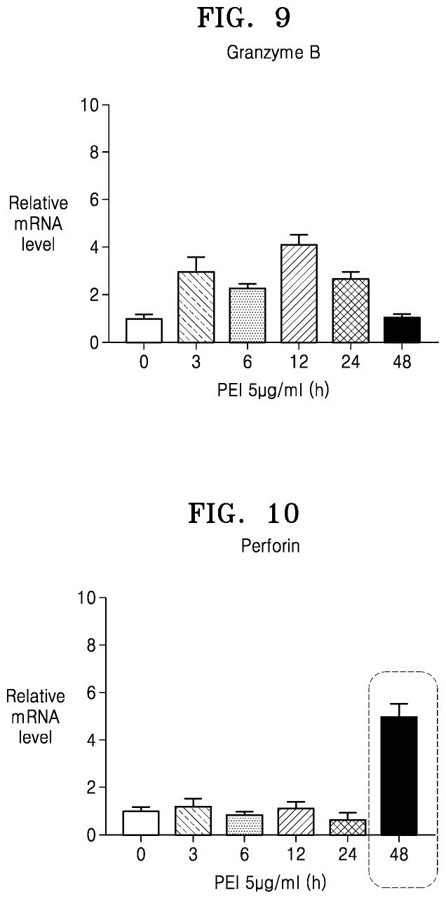

FIG. 9 is a graph showing changes in the expression of granzyme protein as a result of culturing NK cells with 5 μg/ml of polyethylenimine over time.

FIG. 10 is a graph showing changes in the expression of perforin protein as a result of culturing NK cells with 5 μg/ml of polyethylenimine over time.

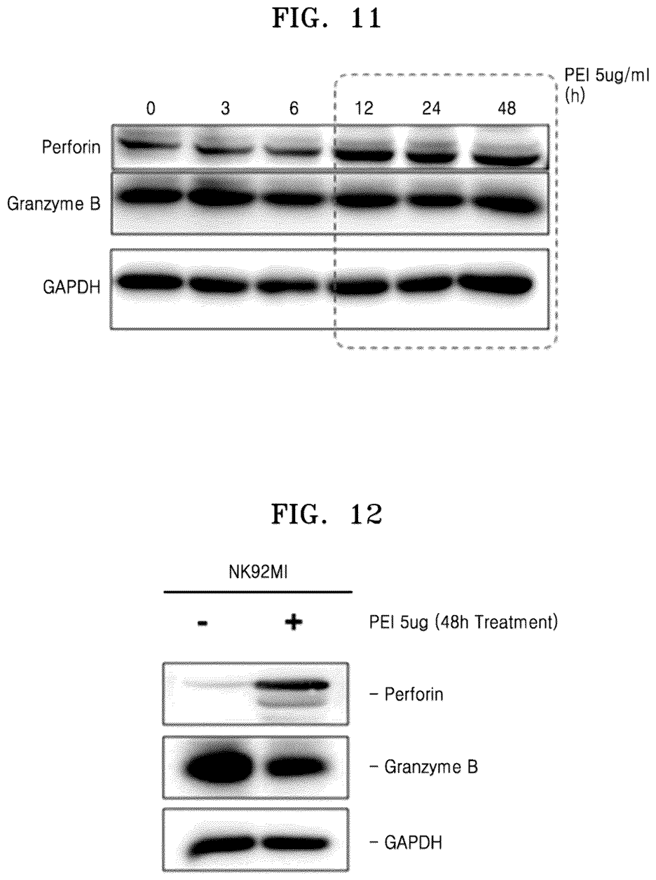

FIG. 11 is an image showing changes in expressions of granzyme protein and perforin protein as a result of culturing NK cells with 5 μg/ml of polyethylenimine over time.

FIG. 12 is an image showing changes in the expression of granzyme protein and perforin protein as a result of culturing NK cells with 5 μg/ml of polyethylenimine for 48 hours.

As a result, as shown in FIG. 12, it was confirmed that the amount of granzyme B protein in the cultured NK cells was not changed, but the amount of perforin protein therein was significantly increased. In particular, as shown in FIGS. 9 to 11, when cultured for 48 hours, it was confirmed that the amount of perforin protein was significantly increased compared to cells cultured for less than 48 hours. These results suggest that polyetheramines induce stabilization of perforin proteins, thereby increasing amounts thereof and, consequently, activation of natural killer cells.

### 6.2 Confirmation of Dependence of Intracellular Perforin Amount on Perforin Stability

In order to confirm whether the increase in the amount of intracellular perforin in Example 6.2 is related to the degree of stabilization of intracellular perforin, the NK cells of Example 1 were treated with MG132, a protease inhibitor, to prevent intracellular protein degradation, and then, cultured. Thereafter, Western blotting was performed on the cultured NK cells to identify the amounts of granzyme B and perforin protein.

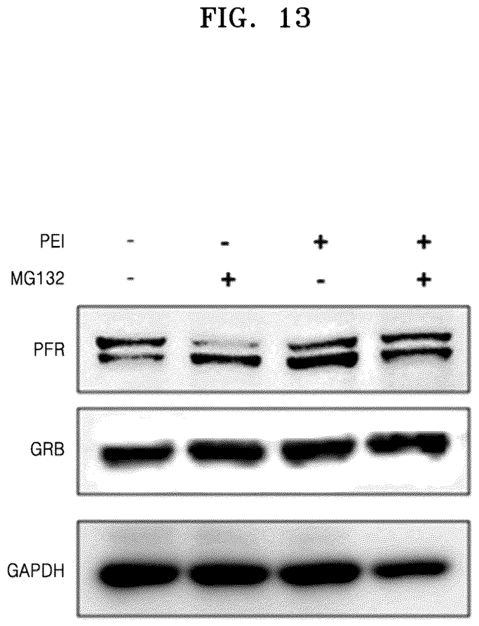

FIG. 13 is an image showing changes in expression of perforin protein as a result of culturing NK cells with polyethylenimine and MG132.

As a result, as shown in FIG. 13, as a result of analyzing the amount of perforin protein after preventing intracellular proteolysis, it was confirmed that the amount of perforin protein was increased by treatment with MG132. These results confirm that the treatment with polyetheramine results in the increase in the stability of protein and, as a result, the amount of intracellular perforin protein is increased so that the amount of perforin protein is not additionally increased by treatment with MG132. Therefore, this indicates that the amount of perforin in NK cells is regulated according to the stability of a protein.

1. A composition for increasing a perforin protein in a cell, the composition comprising a cationic substance as an active ingredient.

2. The composition of claim 1, wherein the cell is an immune cell.

3. The composition of claim 1, wherein the cationic substance is polyethylenimine (PEI).

4. The composition of claim 1, wherein the polyethylenimine has a molecular weight of 10,000 mM to 30,000 mM.

5. The composition of claim 1, wherein the polyethylenimine is branched.

6. The composition of claim 1, wherein an amount of polyethylenimine is 1 μg/ml to 10 μg/ml.

7. A composition for enhancing an activity of immune cell, the composition comprising a cationic substance as an active ingredient.

8. A culture method of promoting an activity of natural killer cells, the culture method comprising culturing immune cells with a cationic substance.

9. A method of preventing or treating cancer, the method comprising administering the composition of claim 1 to a subject.

10. Use of the composition of claim 1, for use in increasing intracellular perforin protein.

11. Use of the composition of claim 1, for use in increasing an activity of an immune cell.

12. Use of a cationic substance, for use in increasing intracellular perforin protein.
